(19) United States
(12) Patent Application Publication
(10) Pub. No.: US 2026/0310949 A1
(43) Pub. Date: Oct. 8, 2026

(54) VECTOR SYMBOLIC ARCHITECTURE (VSA) FOR MULTI-CHANNEL TIMESERIES ANALYSIS

(71) Applicant: International Business Machines Corporation, Armonk, NY (US)

(72) Inventors: Francesco Stefano Carzaniga, Aarau (CH); Michael Hersche, Zürich (CH); Abbas Rahimi, Rüschlikon (CH)

(21) Appl. No.: 19/093,174

(22) Filed: Mar. 27, 2025

(51) Int. Cl. G06N 3/049; G16H 20/10; G16H 50/30

(52) U.S. Cl. CPC G06N 3/049; G16H 20/10; G16H 50/30

(57) ABSTRACT

A plurality of channels are encoded to provide short-term feature extraction from multi-channel timeseries data for each of a plurality of short-term windows to generate channel-by-channel feature vectors and to provide a structured latent space representation of the plurality of channels. The channel-by-channel feature vectors are fused to incorporate an original spatial structure of the plurality of channels into a single short-term representation for each of the plurality of short-term windows. Long-term temporal features are extracted from the single short-term representations and long-term context processing is performed on the long-term temporal features. The multi-channel timeseries data is classified based on results of the long-term processing and a result of the classification is output.

### BACKGROUND

The present invention relates generally to the electrical, electronic and computer arts and, more particularly, to machine learning.

Multi-channel timeseries are collections of data points where one dimension represents time and the other represents a different variable. Bio-signals, such as from extra-cranial electroencephalogram (EEG) or electrocardiogram (ECG), are an example of multi-channel timeseries with the second dimension being the channel (i.e., the signal from the electrode). Multi-channel timeseries also include temperature, humidity, and the like at a location over time.

Time-series processing can be performed both with classical methods (such as autoregressive integrated moving average (ARIMA) and gradient boosting) and neural networks (NNs). Conventional models based on recurrent NNs and convolutional NNs often achieve state-of-the-art results. NN models generally require a fixed number of channels. While transformers do not require a fixed number of channels, they are much larger and less efficient neural network models. Many timeseries, on the contrary, can have a varying size, making such networks inapplicable. For example, EEG traces from different patients typically have different number of channels; fault data from different sources have different number of sensors. Neural networks greatly benefit from large and heterogeneous data sources, which emphasize the limitation of the input size. For the same reason, neural networks are also often not capable of generalizing across tasks and modalities. VSAs represent a promising approach to the problem and have received much attention. Vector symbolic architectures (VSAs) refer to representing data as vectors with high dimensionality (e.g., vectors in the thousands). For example, one architecture involves superposing data vectors into a single high-dimensional vector, allowing for various operations to be performed on the resulting data structure.

### BRIEF SUMMARY

Principles of the invention provide techniques for VSA-based system for vector symbolic architecture (VSA) for multi-channel timeseries analysis. In one aspect, an exemplary method includes the operations of encoding a plurality of channels to provide short-term feature extraction from multi-channel timeseries data for each of a plurality of short-term windows to generate channel-by-channel feature vectors and provide a structured latent space representation of the plurality of channels; fusing the channel-by-channel feature vectors to incorporate an original spatial structure of the plurality of channels into a single short-term representation for each of the plurality of short-term windows; extracting long-term temporal features from the single short-term representations; performing long-term context processing on the long-term temporal features; classifying the multi-channel timeseries data based on results of the long-term processing; and outputting a result of the classification.

In one aspect, a computer program product comprises one or more tangible computer-readable storage media and program instructions stored on at least one of the one or more tangible computer-readable storage media, the program instructions executable by a processor, the program instructions comprising encoding a plurality of channels to provide short-term feature extraction from multi-channel timeseries data for each of a plurality of short-term windows to generate channel-by-channel feature vectors and provide a structured latent space representation of the plurality of channels; fusing the channel-by-channel feature vectors to incorporate an original spatial structure of the plurality of channels into a single short-term representation for each of the plurality of short-term windows; extracting long-term temporal features from the single short-term representations; performing long-term context processing on the long-term temporal features; classifying the multi-channel timeseries data based on results of the long-term processing; and outputting a result of the classification.

In one aspect, an apparatus comprises a memory and at least one processor, coupled to the memory, and operative to perform operations comprising encoding a plurality of channels to provide short-term feature extraction from multi-channel timeseries data for each of a plurality of short-term windows to generate channel-by-channel feature vectors and provide a structured latent space representation of the plurality of channels; fusing the channel-by-channel feature vectors to incorporate an original spatial structure of the plurality of channels into a single short-term representation for each of the plurality of short-term windows; extracting long-term temporal features from the single short-term representations; performing long-term context processing on the long-term temporal features; classifying the multi-channel timeseries data based on results of the long-term processing; and outputting a result of the classification.

As used herein, “facilitating” an action includes performing the action, making the action easier, helping to carry the action out, or causing the action to be performed. Thus, by way of example and not limitation, instructions executing on a processor might facilitate an action carried out by instructions executing on a remote processor, by sending appropriate data or commands to cause or aid the action to be performed. Where an actor facilitates an action by other than performing the action, the action is nevertheless performed by some entity or combination of entities.

Techniques as disclosed herein can provide substantial beneficial technical effects, as will be discussed further below. Features and advantages will become apparent from the following detailed description of illustrative embodiments thereof, which is to be read in connection with the accompanying drawings.

### BRIEF DESCRIPTION OF THE DRAWINGS

The following drawings are presented by way of example only and without limitation, wherein like reference numerals (when used) indicate corresponding elements throughout the several views, and wherein:

FIG. 1A is a flowchart of an exemplary algorithm for a VSA-based system, in accordance with exemplary embodiments;

FIG. 1B is a first block diagram of an exemplary architecture of the channel-adaptive (CA) classifier, in accordance with exemplary embodiments;

FIG. 2 is a second block diagram of an exemplary architecture of the CA classifier, in accordance with exemplary embodiments;

FIG. 3A is a table that identifies the three components of the composable CA architecture, in accordance with exemplary embodiments;

FIG. 3B is a table of the hyper-parameter selection for exemplary embodiments (CA-EEG1 and CA-EEG2), in accordance with exemplary embodiments;

FIG. 3C is a table of the hyper-parameter selection for conventional models, in accordance with exemplary embodiments;

FIG. 3D is a table that summarizes the ablation results for each component on the short-term dataset, in accordance with exemplary embodiments;

FIGS. 4A and 4B illustrate the seizure classification performance of CA models, in accordance with aspects of the invention, on a conventional long-term dataset, such as the Long-term SWEC iEEG Dataset, in accordance with exemplary embodiments;

FIG. 5 illustrates the seizure classification performance of CA models, in accordance with aspects of the invention, on a conventional short-term dataset, such as the Short-term SWEC iEEG Dataset, in accordance with exemplary embodiments;

FIG. 6 illustrates the seizure classification performance of CA models, in accordance with aspects of the invention, on a conventional short-term dataset, such as the Short-term SWEC iEEG Dataset, in accordance with exemplary embodiments;

FIG. 7 illustrates the performance results for various versions of CA-EEG2 vs. the second conventional EEG network, in accordance with exemplary embodiments;

FIG. 8 illustrates that the cosine similarity is uniform at the beginning and then adapts to the functional similarity between the channels after training, in accordance with exemplary embodiments;

FIG. 9 illustrates the flow of holographic reduced representations (HRR), in accordance with exemplary embodiments;

FIG. 10A shows the results using the CA-EEG2 model run on the conventional long-term dataset, in accordance with exemplary embodiments;

FIG. 10B shows the results using the EEG2 model run on the conventional long-term dataset, in accordance with exemplary embodiments; and

FIG. 11 depicts a computing environment according to an embodiment of the present invention.

It is to be appreciated that elements in the figures are illustrated for simplicity and clarity. Common but well-understood elements that may be useful or necessary in a commercially feasible embodiment may not be shown in order to facilitate a less hindered view of the illustrated embodiments.

### DETAILED DESCRIPTION

Principles of inventions described herein will be in the context of illustrative embodiments. Moreover, it will become apparent to those skilled in the art given the teachings herein that numerous modifications can be made to the embodiments shown that are within the scope of the claims. That is, no limitations with respect to the embodiments shown and described herein are intended or should be inferred.

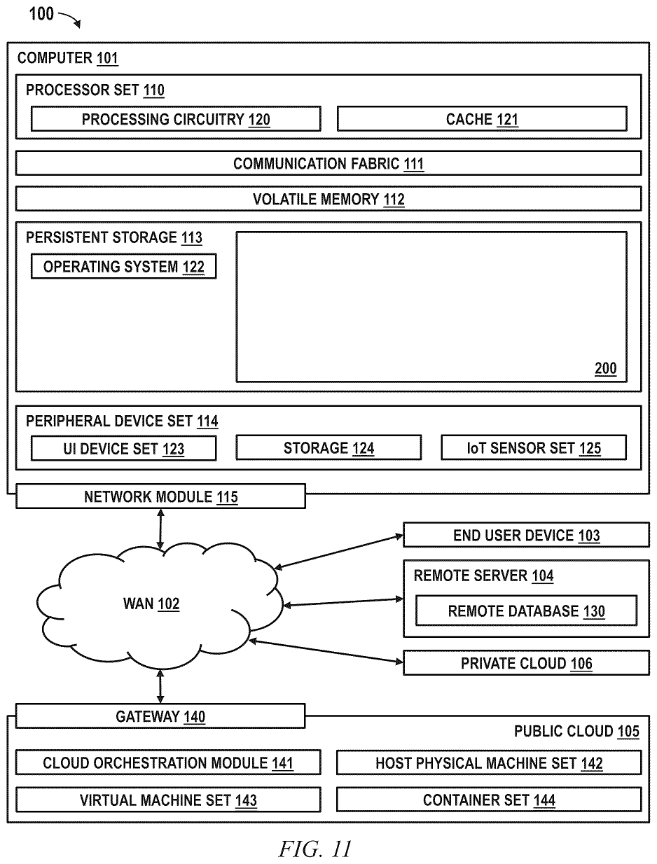

Given the discussion herein (reference characters refer to the drawings discussed below), in aspects of the invention a method comprises encoding a plurality of channels 236 to provide short-term feature extraction from multi-channel timeseries data for each of a plurality of short-term windows to generate channel-by-channel feature vectors 244 and provide a structured latent space representation of the plurality of channels 236; fusing the channel-by-channel feature vectors 244 to incorporate an original spatial structure of the plurality of channels 236 into a single short-term representation for each of the plurality of short-term windows; extracting long-term temporal features from the single short-term representations; performing long-term context processing on the long-term temporal features; classifying the multi-channel timeseries data based on results of the long-term processing; and outputting a result of the classification. Technical benefits include the elimination of the need to train a model from scratch for each subject, thereby significantly decreasing the time and computational resources needed to deploy a fully functioning model. Technical benefits also include enabling adaptation between multi-channel timeseries data having different numbers of channels. In exemplary embodiments, instructions to administer a drug based on the result of the classification are sent over a WAN 102 or a cable (see, FIG. 11).

In exemplary embodiments, the extracting of the long-term temporal features comprises extracting the long-term features by extending an effective receptive field over the plurality of short-term windows. Technical benefits include extending an effective receptive field over the plurality of short-term windows: concatenating short-term windows to handle long-term features.

In exemplary embodiments, each of the plurality of channels 236 is split into the plurality of short-term windows in time and space dimensions prior to the encoding of the plurality of channels 236. Technical benefits include leaving the burden of spatial features to HRR fusion for efficiency and the processing of heterogeneous numbers of channels.

In exemplary embodiments, the encoding of each channel 236 is performed in parallel and independently. Technical benefits include faster encoding.

In exemplary embodiments, the fusing is enabled by holographic reduced representations (HRR) where only a single parameter per channel 236 is used to encode a corresponding channel setup. Technical benefits include parameter and memory efficiency.

In exemplary embodiments, the fusing combines channel-wise features while preserving spatial information between the plurality of channels 236. Technical benefits include enabling adaptation between multi-channel timeseries data having different numbers of channels.

In exemplary embodiments, the plurality of short-term representations are aggregated into a single long-term representation of a long-term context window, and the extracting of the long-term temporal features is based on the single long-term representation. Technical benefits include the efficient handling of long-term representations by first generating short-term representations.

In exemplary embodiments, the fusing of the channel-by-channel feature vectors 244 further comprises preserving a relationship between the channels 236 as an angular distance between components of the single representation. Technical benefits include improved accuracy of the machine learning model.

In exemplary embodiments, a medical condition of a patient is detected based on the classifying of the multi-channel timeseries data, wherein the multi-channel timeseries data is biometric data; and a medical therapy is implemented based on the detected medical condition. Technical benefits include treating medical conditions, such as seizures.

In exemplary embodiments, the implementing of the medical therapy further comprises at least one of automatically delivering a drug therapy based on the detected medical condition and automatically administering responsive neurostimulation based on the detected medical condition. Technical benefits include treating medical conditions and delivering drug therapies and/or neurostimulation based on detected medical conditions.

In exemplary embodiments, the long-term context processing is performed using a temporal convolutional network. Technical benefits include an improved way of performing long-term context processing.

In one aspect, a computer program product comprises one or more tangible computer-readable storage media and program instructions stored on at least one of the one or more tangible computer-readable storage media, the program instructions executable by a processor, the program instructions comprising encoding a plurality of channels 236 to provide short-term feature extraction from multi-channel timeseries data for each of a plurality of short-term windows to generate channel-by-channel feature vectors 244 and provide a structured latent space representation of the plurality of channels 236; fusing the channel-by-channel feature vectors 244 to incorporate an original spatial structure of the plurality of channels 236 into a single short-term representation for each of the plurality of short-term windows; extracting long-term temporal features from the single short-term representations; performing long-term context processing on the long-term temporal features; classifying the multi-channel timeseries data based on results of the long-term processing; and outputting a result of the classification. Technical benefits include the elimination of the need to train a model from scratch for each subject, thereby significantly decreasing the time and computational resources needed to deploy a fully functioning model.

In one aspect, an apparatus comprises a memory and at least one processor, coupled to the memory, and operative to perform operations comprising encoding a plurality of channels 236 to provide short-term feature extraction from multi-channel timeseries data for each of a plurality of short-term windows to generate channel-by-channel feature vectors 244 and provide a structured latent space representation of the plurality of channels 236; fusing the channel-by-channel feature vectors 244 to incorporate an original spatial structure of the plurality of channels 236 into a single short-term representation for each of the plurality of short-term windows; extracting long-term temporal features from the single short-term representations; performing long-term context processing on the long-term temporal features; classifying the multi-channel timeseries data based on results of the long-term processing; and outputting a result of the classification. Technical benefits include the elimination of the need to train a model from scratch for each subject, thereby significantly decreasing the time and computational resources needed to deploy a fully functioning model.

Techniques as disclosed herein can provide substantial beneficial technical effects. Some embodiments may not have these potential advantages and these potential advantages are not necessarily required of all embodiments. By way of example only and without limitation, one or more embodiments may provide one or more of:
a channel-adaptive (CA) architecture that seamlessly functions on any multi-variate signal with an arbitrary number of channels;
each CA-model can be pre-trained on a large corpus of intracranial electroencephalography (iEEG) recordings from multiple heterogeneous subjects, and then finetuned to each subject using equal or lower amounts of data compared to existing state-of-the-art models (and in only ⅕ of the time);
a CA architecture that integrates all the channels of an arbitrary iEEG recording while implicitly preserving the spatial structure of the signal;
a CA architecture that is a drop-in replacement for existing seizure classification models;
elimination of the need to train a model from scratch for each subject, thereby significantly decreasing the time and computational resources needed to deploy a fully functioning model via the steps of encoding a plurality of channels to provide short-term feature extraction from multi-channel timeseries data for each of a plurality of short-term windows to generate channel-by-channel feature vectors; fusing the channel-by-channel feature vectors to incorporate an original spatial structure of the plurality of channels into a single short-term representation for each of the plurality of short-term windows; and extracting long-term temporal features from the single short-term representations;
ability to reuse large parts of the pretrained network modules for new patients where the system can be adapted to handle the number of channels obtained for the new patient and the architecture can then be fine-tuned with very little data;
the ability to pre-train on larger heterogeneous datasets which are more widely available;
ability to process longer context windows of multi-channel timeseries data;
a fusion-enabled encoder that allows training on any dataset and fuses an arbitrary number of channels into a single representation, which surpasses the fixed-channel setup limitation;
a fusion-enabled encoder that allows for training with larger datasets composed of multiple heterogeneous subjects with different spatial setups and number of channels, which surpasses the limited data sources limitation;
a fusion schema that is enabled by holographic reduced representations (HRR), and allows exemplary models to be used across different subjects and clinical setups without any modifications to the architecture;
a temporal memory to increase the effective context size;
seamless integration of existing state-of-the-art models, with no major limitations to compatibility with future models;
the union of multiple short signal windows to generate an integrated classification over minutes of a signal, which surpasses the short context window limitation;
the use of two levels of processing-one short-term and another aggregate long-term-to greatly increase the context window with minimal impact to the model size and complexity, which makes seizure detection possible at a clinically relevant scale of up to minutes of a signal;
a personalized model that can be deployed with 5× fewer epochs of training in comparison to a full re-training;
efficient techniques for processing any multi-variate timeseries data, such as weather data, fault analysis (including predictive failure analysis), financial data, and the like, and to support deep learning for timeseries; and
a system that is generally much smaller than transformers (a few million parameters vs. billions of parameters).

Multi-variate timeseries are one of the primary data modalities involved in large classes of problems, where deep learning models represent the state-of-the-art solution. In the healthcare domain, electrophysiological data, such as intracranial electroencephalography (iEEG), is used to perform a variety of tasks. However, iEEG models require that the number of channels be fixed, while iEEG setups in clinics are highly personalized and thus vary considerably from one subject to the next. To address this concern, a channel-adaptive (CA) architecture that seamlessly functions on any multi-variate signal with an arbitrary number of channels is disclosed. Each CA-model can be pre-trained on a large corpus of iEEG recordings from multiple heterogeneous subjects, and then finetuned to each subject using equal or lower amounts of data compared to existing state-of-the-art models, and in only 20% of the time. The disclosed CA-models are evaluated on a seizure detection task, both on short-term (~20 hours) and long-term (~2500 hours) datasets. In particular, CA-EEG1—based on a first conventional EEG network—is trained on a single seizure of the tested subject, while the baseline first conventional EEG network is trained on all but one. CA-EEG1 surpasses the baseline in median F1-score (0.78 vs 0.76). Similarly, CA-EEG2—based on a second conventional EEG network—also surpasses its baseline (0.79 vs 0.74). (EEGWaveNet is a non-limiting example of the first conventional EEG network (see, P. Thuwajit, P. Rangpong, P. Sawangjai, P. Autthasan, R. Chaisaen, N. Banluesombatkul, P. Boonchit, N. Tatsaringkansakul, T. Sud-hawiyangkul, and T. Wilaiprasitporn, “EEGWaveNet: Multiscale CNN-based spatiotemporal feature extraction for EEG seizure detection,” IEEE Transactions on Industrial Informatics, vol. 18, no. 8, 2022). EEGNet is a non-limiting example of the second conventional EEG network (see, V. J. Lawhern, A. J. Solon, N. R. Waytowich, S. M. Gordon, C. P. Hung, and B. J. Lance, “EEGNet: a compact convolutional neural network for EEG-based brain-computer interfaces,” Journal of Neural Engineering, vol. 15, no. 5, 2018).) Overall, it is shown that the CA architecture is a drop-in replacement for existing seizure classification models, bringing better characteristics and performance across the board.

### INTRODUCTION

Multi-variate timeseries are a ubiquitous source of data, generated and processed every day in many research and industry environments. Deep learning (DL) represents one of the most successful methods for analyzing such large quantities of data, and has been widely adopted.

In the medical domain, DL models have been used to solve a variety of tasks involving electroencephalograms recorded non-invasively with extra-cranial electrodes (EEG) signals, and invasively with intracranial electrodes (iEEG). EEG signals are often recorded in hospitals for diagnostic purposes, but their use extends into research and commercial environments as well. As an example, one of the most compelling uses of iEEG in the clinic is the detection of seizures and the precise localization of seizure onset regions in subjects suffering from pharmaco-resistant epilepsy. However, EEG data remains highly challenging for DL models. In particular, the DL models need to be trained from scratch for each subject and are not capable of generalizing to unseen clinical recording setups, such as different numbers of channels and electrode locations. Large transformer-based models have partially addressed this limitation, but their size and the resources necessary to train and deploy them make them impractical for clinical use. Moreover, the amount of data that these models consider when evaluating the occurrence of a seizure, i.e., the context window, is severely restricted when compared to that of a human expert. This limits their overall performance. In practice, these limitations make such models burdensome to apply for general real-world use and have hindered their widespread adoption. Three main issues affecting DL models for multi-variate timeseries in general are highlighted, emphasizing their role in EEG.

### Fixed Channel Setup

First, DL models often need a fixed number of channels to properly process the timeseries recordings. Moreover, the setup of the channels also needs to remain fixed, as the models cannot easily generalize to unseen configurations. Neither of these two requirements can be satisfied across heterogeneous subjects, whose recording electrodes are personalized and thus often vary in number and location. This issue is especially relevant for iEEG recordings, since their setup is guided by clinical needs only.

Nonetheless, iEEG remains the preferred data source for training DL models due to its higher signal-to-noise ratio. Thus, DL models are often not capable of generalizing across heterogeneous subjects and need to be trained from scratch for each subject. This significantly increases the time and computational resources needed to deploy a fully functioning model.

### Limited Data Sources

Second, by training only on the data from a single subject, current DL models are precluded from using the large amounts of EEG signals that have been already collected. Typically, EEG signals are collected by placing electrodes on the scalp of the subject. Therefore, such EEG recordings are not invasive, and they are commonplace in many institutions, so EEG data is abundant. The availability of EEG data is also expected to increase significantly, given the recent developments of wearable EEG systems for ultra long-term recordings. However, non-invasively recorded EEG suffers from limited signal-to-noise ratio and low spatial and temporal resolutions. iEEG surpasses these limitations by implanting electrodes directly onto or even into the subject's brain. As a consequence, iEEG is markedly more invasive and its availability is limited. Therefore, the overall performance of DL models on iEEG suffers as a consequence of this limited availability, and would greatly benefit from being able to pre-train on larger heterogeneous datasets which are more widely available.

### Short Context Window

Third, when detecting seizures in an iEEG signal, current DL models use context windows on the order of a few seconds to reduce their computational complexity. In contrast, human experts often analyze minutes of data in order to make a decision. This limits the scale at which DL models can operate, and makes them unable to properly assess more unusual seizure presentations, which can last for multiple seconds to minutes or be closely matched to the near past signal. Once again, the seizure detection performance suffers as a consequence.

To address the limitations outlined above and bring DL models closer to practical application in the clinic, one or more embodiments advantageously employ a channel-adaptive (CA) architecture. The CA architecture includes a fusion-enabled encoder to allow training on any dataset, and a temporal memory to increase the effective context size. In particular, it combines:
fusion of an arbitrary number of channels into a single representation, which surpasses the fixed-channel setup limitation (the fusion also allows for training with larger datasets composed of multiple heterogeneous subjects with different spatial setups and number of channels, which surpasses the limited data sources limitation);
union of multiple short signal windows to generate an integrated classification over minutes of signal, which surpasses the short context window limitation; and
seamless integration of existing state-of-the-art models, with no limitations to compatibility with future models.

An exemplary CA architecture in accordance with one or more embodiments integrates all the channels of an arbitrary iEEG recording while implicitly preserving the spatial structure of the signal. In exemplary embodiments, a fusion schema is enabled by holographic reduced representations (HRR), and allows the exemplary model to be used across different subjects and clinical setups without any modifications to the architecture. (The skilled person will be familiar with holographic reduced representations from, for example, T. Plate, “Holographic reduced representations,” IEEE Transactions on Neural Networks, vol. 6, no. 3, 1995 and, given the teachings herein, can adopt known holographic reduced representations to implement aspects of the invention.) Using HRR, a learnable and maximally efficient fusion of the channels is developed, with only a single parameter per channel to encode the channel setup. Moreover, the exemplary architecture uses two levels of processing-one short-term and another aggregate long-term-to greatly increase the context window with minimal impact to the model size and complexity, which makes seizure detection possible at a clinically relevant scale of up to minutes of signal. The recognition of a temporal pattern with a long context window is enabled by the use of temporal convolutional networks (TCNs) to provide long-term context processing. The characteristics of the exemplary CA architecture are made use of by, for example:
pre-training on a large multi-subject corpus of iEEG recordings with 32-128 electrodes;
fine-tuning on a few seizures for a specific subject; and
deploying the personalized model with 5× fewer epochs required by a full re-training, and achieving state-of-the-art performance.

This training schema increases the amount of data available by 5 to 15 times, depending on the pre-training setup. Finally, the exemplary model achieves state-of-the-art results in the seizure detection task even in a challenging realistic scenario, where the disclosed model only has access to a single subject-specific seizure. This is in contrast to existing models that require multiple subject-specific seizures to train effectively.

### Channel Fusion in Bio-Signal Classification

Channel fusion has been used for the classification of bio-signals using fixed bipolar key vectors and variable value vectors. (The skilled person will be familiar with channel fusion from, for example, A. Burrello, K. Schindler, L. Benini, and A. Rahimi, “One-shot learning for iEEG seizure detection using end-to-end binary operations: Local binary patterns with hyperdimensional computing,” in 2018 IEEE Biomedical Circuits and Systems Conference (BioCAS), 2018; and A. Moin, A. Zhou, A. Rahimi, A. Menon, S. Benatti, G. Alexandrov, S. Tamakloe, J. Ting, N. Yamamoto, Y. Khan et al., “A wearable biosensing system with in-sensor adaptive machine learning for hand gesture recognition,” Nature Electronics, vol. 4, no. 1, 2021. Given the teachings herein, the skilled person can adopt known channel fusion techniques to implement aspects of the invention.) The use of fixed bipolar vectors, however, is non-differentiable and limits their flexibility when the number and the location of the electrodes vary considerably. In contrast, in exemplary embodiments, a fully differentiable, learnable schema is used with only one parameter per channel to represent the relevant information content of the spatial dimension.

In exemplary embodiments, a system includes a composable neural network architecture which can efficiently operate on varying-size multi-channel timeseries data and classify multi-channel timeseries with high accuracy. The system analyzes multi-channel timeseries whereby:
a set of neural network controllers independently process univariate time-series;
a VSA-based fusion technique combines processed features from an arbitrary number of channels into composite vectors which also contain spatial information (only one sparse matrix needs to be learned); and
a decision making process that extracts long term features by extending the effective receptive field over multiple short-term windows (controller and decider form a two-tiered system where multiple short windows are considered in parallel to extend the size of the signal used to make a decision).

An exemplary system according to one or more embodiments provides efficient techniques to process any multi-variate timeseries, such as weather data, fault analysis (including predictive failure analysis), financial data, and the like, and to support deep learning for timeseries, such as via conventional transformers. The system is generally much smaller than transformers (a few million parameters vs. billions of parameters).

### Channel-Adaptive (CA) Architecture

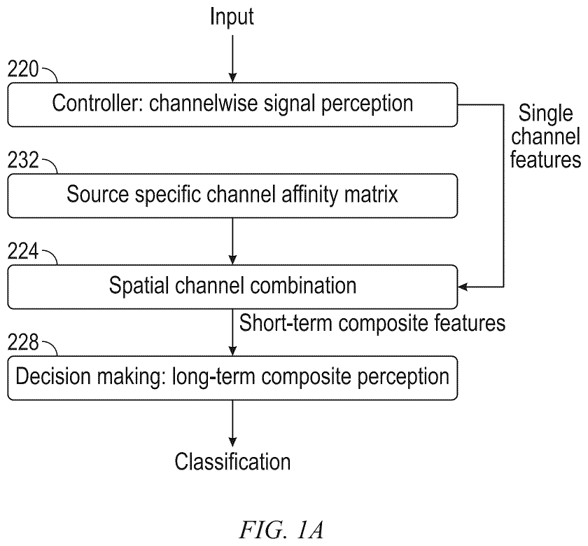

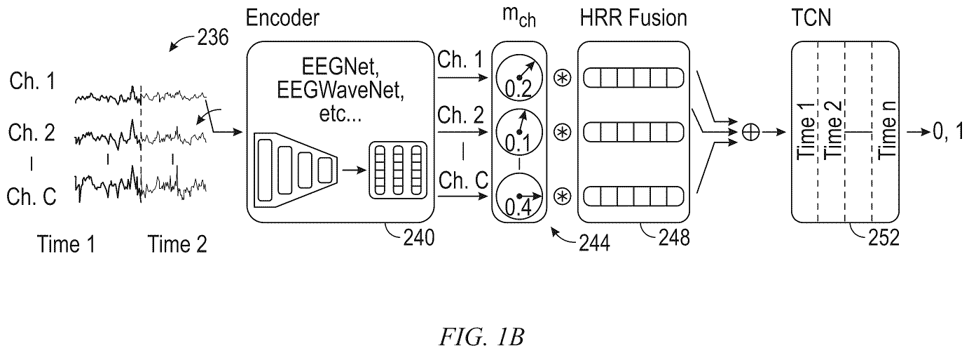

FIG. 1A is a high-level flowchart of an exemplary algorithm for a VSA-based system, in accordance with exemplary embodiments. In exemplary embodiments, a controller 220 processes one or more channels to provide channel-wise signal perception. (In exemplary embodiments, the controller 220 is implemented in software and maps the temporal input signals to a feature representation (similar to an embedding function).) A channel memory 232 (implemented with data structures stored in a computer memory) stores a source-specific channel affinity matrix. The affinity matrix can be as compact as just storing one single value per channel. The channel affinity matrix is then used by spatial channel combination software 224 to combine the single channel features into a composite feature representation representing short-term behavior. Finally, the feature representation is then fed into a perceptron (decision making software 228) that can recognize long-term patterns.

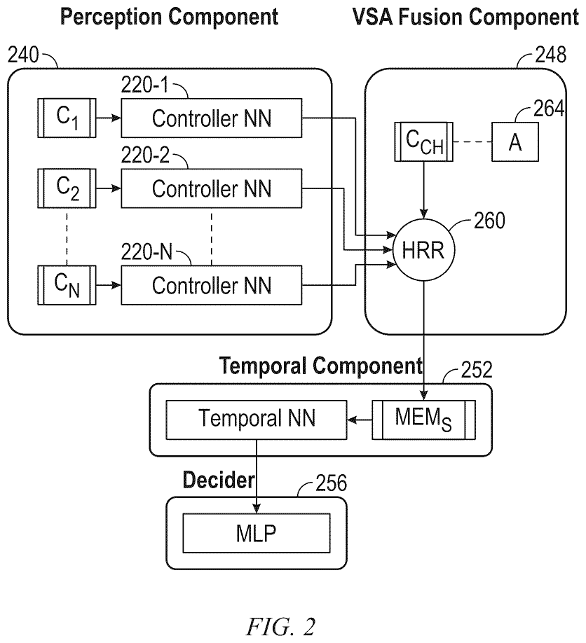

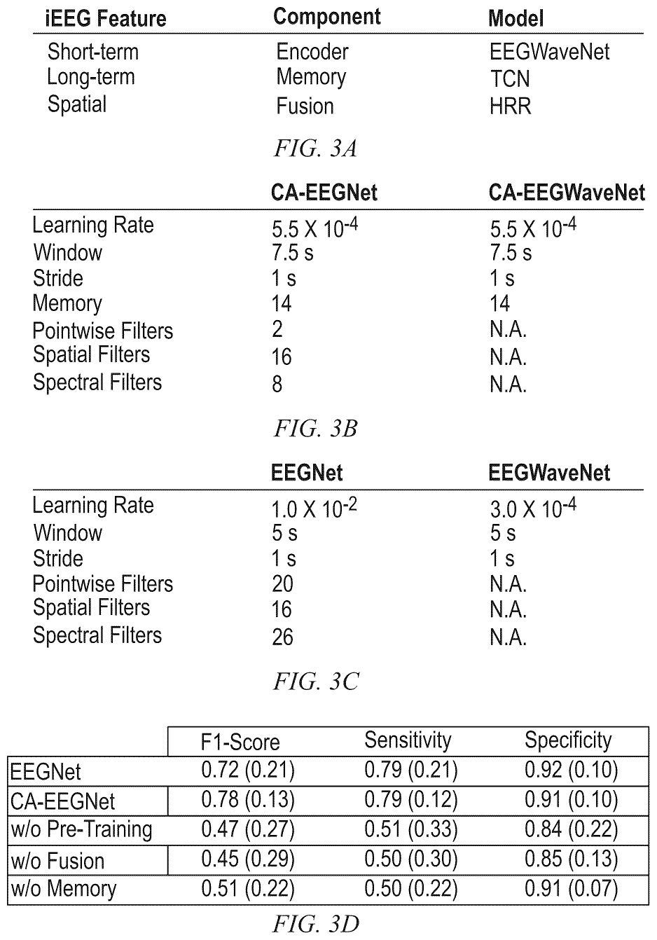

The exemplary CA architecture features interchangeable base components (see FIGS. 1B and 2) and combines an arbitrary number of channels while preserving the relevant spatial information content, thus enabling channel-adaptive training. Three distinct and intrinsic characteristics of an iEEG signal that can help identify a seizure are identified and a component of the exemplary architecture is associated with each characteristic. FIG. 3A is a table that identifies the three components of the composable CA architecture, in accordance with exemplary embodiments. Each component attends to a different aspect of the signal. Three features of interest are identified: short-term, long-term, and spatial. Each feature is assigned a best-performing model.

FIG. 1B is a first block diagram of an exemplary architecture of the CA classifier, in accordance with exemplary embodiments. The classifier includes three components. The software encoder 240 component acts channel-wise and extracts short-term temporal features from each channel 236. A software-based holographic (HRR) fusion component 248 combines the channel-wise features 244 while preserving the spatial structure. Finally, the memory component (TCN) 252 extracts the relevant long-term temporal features and outputs the classification decision.

In exemplary embodiments, an EEG signal X∈R^{C×T} has C channels 236 and a duration T=t×fₛ of t seconds at a sampling frequency of fₛ. The signal is first divided into short windows (on the order, for example, of seconds) and then fed to the encoder component 240 channel-by-channel to extract short-term features. The channel-by-channel feature vectors 244 are fused together through an exemplary HRR-based learnable schema to recover the original spatial structure of the signal while producing a single representation for further processing. Finally, multiple short-term fused representations are aggregated into a long-term context window, which the memory component 252 uses to produce the final classification.

A composable approach is chosen to allow the reuse of the architecture following the development of newer encoder or memory models, to preserve the channel-adaptive training schema and the fusion interface. To identify the exemplary CA model from its components, CA is prefixed to the name of the encoder model. In particular, CA-EEG1 and CA-EEG2 are considered.

### Encoder

First, the signal (X) is divided channel-wise into short patches

$${x}_{i , j} ∈ {R}^{W} , i ∈ 1 , … , C , j ∈ 1 , … , \frac{T}{W} ,$$

with the patch size W on the order of, for example, a few seconds. The patches serve as the input to the encoder model, which has the objective of extracting temporal features on the short time scale. In particular, the encoder model is replaceable as improved solutions emerge, as long as it provides some output features p_{i,j}∈R^{d}. This allows the architecture to remain the same, but to include the benefits of different models at the same time. In fact, this composability is showcased by testing the CA classifier using the first and second conventional EEG networks and as the encoder models. To handle an arbitrary number of channels 236, the encoder 240 processes each x_{i,j} independently into an output vector p_{i,j}, ignoring any inter-channel interaction. Thus, the specific iEEG setup can be adapted to and will process the spatial information content of the signal in the downstream fusion component 248.

FIG. 2 is a second block diagram of an exemplary architecture of the CA classifier, in accordance with exemplary embodiments. In exemplary embodiments, an encoder 240 splits the signal in both time and space dimensions where each channel is divided into short windows, which are then used as inputs to the NN model of a corresponding controller NN 220-1, 220-2, . . . , 220-N to extract relevant features (each channel 236 is processed independently and in parallel with all others). In a non-limiting example, the neural network is a convolutional recurrent linear neural network.

The fusion component 248 fuses the channel-by-channel feature vectors 244 produced by the controller NN 220-1, 220-2, . . . , 220-N using an HRR mechanism 260 to incorporate an original spatial structure of the plurality of channels 236 into a single representation, which surpasses a fixed-channel setup limitation. Feature vectors of multiple time windows are accumulated in the memory component 252 and processed by the temporal neural network of the memory component 252, as described more fully below. A final classification is generated by a multilayer perceptron (MLP) decider 256. Diagonal matrix A 264 is discussed further below.

The computation becomes embarrassingly parallel in the number of channels, severely reducing computational complexity and becoming particularly amenable to hardware implementation. This late fusion approach allows bypassing the usual uniformity requirement of convolutional neural networks (CNNs) in the size and number of dimensions.

### Fusion

In one or more embodiments, the objective of the fusion component 248 is to integrate the spatial information content of the signal. Given input feature vectors p_{i,j}, a goal is to construct for each time point a single integrated representation fⱼ∈ [symbol] , j∈{1, . . . , TW} which contains the needed information. To preserve the spatial interactions between the channels 236, a fully trainable fusion schema based on holographic reduced representations (HRR [18]) was developed, a family of vector symbolic architectures. The main steps of the fusion component 248 are presented below.

First, a random unitary (in C) basis vector v∈ [symbol] is chosen. Then, the relationship between the channels 236 is tracked through a learnable vector m_{ch}∈ [symbol] , with one value per channel. m_{ch} 244 is a maximally efficient representation of the spatial information contained in the iEEG signal. The coefficients of m_{ch} indicate the angle of rotation of the basis vector in Fourier space, i.e.

$$rot ( v , r ) = {ℱ}^{- 1} ( {F ( v )}^{r} ) , \qquad ( 1 )$$

where the element-wise power of each component of [symbol] (v) by an angle r from 0 to 1 is taken (which indicates a rotation by 2π radians). In particular, the closer the m_{ch} values of two channels 236 are, the stronger the relationship between them. To obtain the final integrated vector across all channels, the HRR schema is applied:

$${f}_{j} = {\sum }_{i}^{C} {p}_{i , j} \text{?} rot ( v , {m}_{c h}^{i} ) \qquad ( 2 )$$

where [symbol] is the circular convolution acting as the binding operator. The HRR schema ensures that similar channels 236 constructively interfere in the latent space while different channels 236 do not interfere, thanks to the binding that maps them to separate protected subspaces. Moreover, the relationship between the channels 236 is preserved as the angular distance between the components of fⱼ, which can then be processed by the downstream components. Therefore, through the fusion component 248, any number of channels 236 can be processed without loss of generality or representation power.

In exemplary embodiments, VSAs based on fractional power encoding (FPE) is disclosed to autonomously build a latent space which is consistent with spatial structure. The exemplary system autonomously learns a set of combination vectors for VSA based on the functional similarity of the channels. FPE combines Fourier holographic reduced representation (fHRR) with explicit similarity specifications {x₁, . . . , xₙ}.

In exemplary embodiments, a complex unitary seed vector V is chosen at random. Then, to produce a vector with distance x₁, the vector is moved to Fourier space and its power of 1+x₁ is taken. This results in a rotation of V periodic w.r.t. x₁. This operation is repeated for all required similarities and a set of vectors {V₁, . . . , Vₙ} with the desired properties is obtained. (w.r.t.=with respect to.)

For each model, a diagonal matrix A 264 is initialized with ones to indicate to the network that all channels 236 are equal at the beginning of training. As training progresses, matrix A 264 is populated by values based on the similarity scores that the network autonomously reconstructs from the data where |Aᵢ−Aⱼ| is the similarity between channels i and j. Matrix A 264 is then used to generate the above mentioned set of vectors with FPE where |Aᵢ−Aⱼ|*360 is the angle between encoding vectors vᵢ and vⱼ. The outputs of the controller are convolved (bound) channel-wise with the generated set of encoding vectors to preserve their spatial location finally adding (bundling) them together to provide a single output:

$$Out = {\sum }_{i} f ( {X}_{i} ) ∘ {v}_{i}$$

FIG. 8 illustrates that the cosine similarity is uniform before training and then after training adapts to the functional similarity between the channels, in accordance with exemplary embodiments.

### Fractional Power Encoding

Vector symbolic architectures (VSAs) are symbolic models that use vector representations to build semantically meaningful subspaces of [symbol] . The vectors used in VSAs are usually drawn randomly and i.i.d. (Independent and Identically Distributed), such that a large number of them can be drawn without interfering with each other. This translates to the vectors in the collection having low similarity (e.g., cosine similarity) and is guaranteed by the concentration of measure as d becomes larger. To build the aforementioned subspaces, the vectors are combined through two elementary operations: binding and bundling. On one hand, binding serves to build an output vector which is dissimilar to the input vectors used in its creation. On the other hand, bundling combines multiple vectors into one which is similar to all its components.

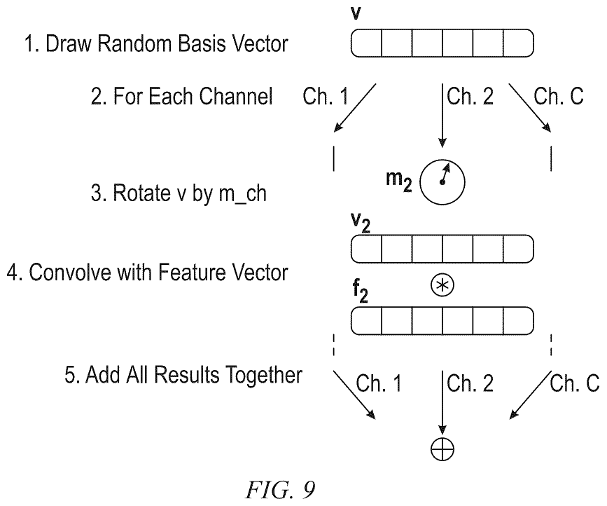

VSA models are characterized mainly by these two properties: the random vectors and the elementary operations. In exemplary embodiments, fractional power encoding (FPE) is used together with holographic reduced representations (HRR). The suitability of this VSA model for the fusion component of the disclosed channel-adaptive classifier is analyzed (see FIG. 9).

FPE is used to efficiently track the relationship between the spatial channels of an iEEG signal and generate appropriate symbolic vectors. Consistent with the HRR paradigm, circular convolution ( [symbol] ) is also chosen to be the binding operation, and simple summation to be the bundling operation. Circular convolution can be efficiently implemented by considering it as multiplication in Fourier space, such that

$$v \text{?} w = {ℱ}^{- 1} ( ℱ ( v ) ⊙ ℱ ( w ) ) ,$$

where ⊙ is elementwise multiplication. Using FFT, [symbol] becomes computationally very lightweight.

Next, this binding operation is applied to generate FPE key vectors. Given a random vector v and an angle r, v is rotated by 2π×r radians by self-binding r times as follows:

$$rot ( v , r ) = v \text{?} … \text{?} v = {ℱ}^{- 1} ( {ℱ ( v )}^{r} ) ,$$

where the [symbol] operation is repeated r times.

It is to be to ensured that each rotated vector remains on the unit sphere, i.e., it is only different in its angle to another vector, to avoid unduly biasing the overall model towards one specific angle. However, rot( ) does not necessarily preserve the norm of v. To fix this issue, v should first be made unitary, i.e. normalize it such that

$$‖ {ℱ ( v )}_{i} ‖ = 1 ∀ i$$

The required structure is capable of fusing the channels and preserving their spatial relationship. As before, m_{ch} is a vector containing angles, each associated to a specific channel. To avoid possible issues with different basis vectors v, as rot (·, 0) produces the same result for any vector, m_{ch} is constrained to have values from 1 to 2. Subtraction by 1 is then performed in rot to recover the angle interpretation. The outputs of the encoder component are the channel value vectors pᵢ, which are first bound with the FPE channel key vectors and finally bundled as follows

$$f = {\sum }_{i} {p}_{i} \text{?} rot ( v , {m}_{c h}^{i} )$$

One such fused vector is obtained for each time window in the signal. The convolutional binding operator is distributive with respect to addition, meaning that similar key vectors constructively interfere. In contrast, dissimilar key vectors produce dissimilar results, such that the dissimilar channels do not interact in the bundled vector. Moreover, each subject has its own m_{ch}, since the spatial configuration of the electrodes is unique.

The resulting kernel from this VSA realization approximates the sinc function, therefore the relationship between the channels decays symmetrically as the difference in the angles gets larger. In addition, m_{ch} is learned by the network and translates well to the spatial structure of the electrodes. This can be seen by plotting the cosine similarity of the FPE key vectors at the beginning and at the end of training. In summary, this schema provides a maximally economical (only one value per channel) representation of the spatial information content of an iEEG signal.

### Memory

The encoder 240 and fusion component 248 extract the required spatial short-term temporal information. However, neurologists take into consideration long-term temporal dependencies on the order of minutes when making their decisions regarding seizures. Therefore, it is believed that an effective seizure detection architecture must obtain long-term temporal information as well. To do this, the exemplary CA classifier is equipped with a stack that collects multiple integrated patches fⱼ to form a long-term memory {fⱼ, . . . , f_{j+M}} with, for example, length M=14. The context available to the exemplary network is effectively increased from W to W×M. In exemplary embodiments, a patch length of 7.5 seconds(s) is used to yield an effective context length of 105 s. The memory stack is processed using a temporal convolutional network (TCN), which avoids the computational pitfalls of traditional recurrent neural networks. The TCN of the memory component 252 maintains high efficiency while processing long sequences of iEEG signal.

In exemplary embodiments, the FPE-composite feature vectors of multiple time windows are accumulated in the memory 252. This results in a two-tiered system where the controller is responsible for extracting short-term features, while the decision making can look across these short-term features to make a long-term decision. A temporal convolutional network (TCN) of the memory component 252 is used as an efficient non-recurrent model which cross-analyses these windows, finding similarities and differences to discover anomalies in the lookback window. The TCN processes all sub-windows and, finally, the output indicates the classification result in the larger lookback window.

### Training Setup

In an exemplary approach, all the CA models are trained following a two-stage approach. First, pre-training is performed, then fine-tuning is performed. To assess the performance of the exemplary CA classifier, training and testing on all the subjects were performed with a rigorous procedure, by fine-tuning each model in a leave-one-out cross-validation manner.

### Pre-Training

The exemplary CA model was pretrained on a large amount of heterogeneous data to increase its ability to generalize across subjects. For each testing subject in both the conventional long-term and short-term datasets, a separate model was pretrained on a number of subjects. It is pertinent to note that in one or more embodiments, the subject the model is trained for is excluded from the pre-training selection, and is only used during fine-tuning and testing. In particular, pre-training on 5, 10, and 15 subjects is performed. As data is collected from multiple subjects, the model can be trained with optimal data efficiency and does not suffer from a lack of data availability. Moreover, the selection of subjects is random to avoid bias. The model is pre-trained until the training F1-score plateaus, for a minimum of 25 and a maximum of 50 epochs. To guarantee uniform training coverage, in one or more embodiments, data is selected from a random pre-training subject at every batch. Depending on the number of pre-training subjects and the hardware, this stage can last from a few hours to a few days. On a conventional graphical processing unit (GPU), a maximum pre-training time of <6 hours is achieved, with 15 subjects pre-training.

### Fine-Tuning

In the example, during the fine-tuning stage, only the testing subject was considered. Some of their seizures are fed to the model, mainly to train the spatial fusion component, which is subject-dependent. There are two fine-tuning strategies:
leave-one-out cross-validation (LOOC), where each model is fine-tuned on all but one seizure of the testing subject, resulting in a number of sub-models equal to the number of seizures of the subject; and
leave-all-but-one-out cross-validation (LABOC), where each model is fine-tuned on only one seizure of the testing subject, resulting again in a number of sub-models equal to the number of seizures of the subject.

This guarantees the validity of the disclosed results in a variety of conditions, from having equivalent data to the baselines to having the least amount of data possible. Each model is fine-tuned for a maximum of 10 epochs.

As a baseline, the first and second conventional EEG network models are also trained following their respective original training schema, using the LOOC regime without pre-training. In fact, the first and second conventional EEG networks cannot be trained using the LABOC schema, as the amount of data is insufficient and the performance is poor. In contrast, the exemplary CA models perform at the state-of-the-art even with such a limited amount of subject-specific data.

### Parameters

To obtain the best performing models, both for the exemplary CA models and the two baselines, a round of hyper-parameter optimization was performed with each architecture. For both CA-EEG1 and CA-EEG2, five random subjects are selected for pre-training and two more random subjects are selected for validation. For the baseline models, all but one of the seizures of one random subject are selected for training, and the other is left for validation. In every instance at least 100 hyper-parameter combinations were tested and the one with highest validation F1-score was chosen. The optimization is performed on the short-term dataset, and carried over to the long-term dataset.

It is worth noting that the exemplary CA architecture offers the best performance with the same set of hyper-parameters for both encoder choices. The resulting sizes for the baselines models are 2 million (M) parameters and 227,000 parameters for the second EEG network and the first conventional EEG network, respectively. For the exemplary CA models, the sizes are 1.7M and 995,000 for CA-EEG2 and CA-EEG1, respectively. FIG. 3B is a table of the hyper-parameter selection for exemplary embodiments (CA-EEG1 and CA-EEG2), in accordance with exemplary embodiments. FIG. 3C is a table of the hyper-parameter selection for conventional models, in accordance with exemplary embodiments.

### Conventional Short-Term Datasets

The conventional short-term iEEG dataset contains 16 subjects, 14 hours of recording, and 104 ictal events. Each ictal event is accompanied by three minutes of pre-ictal and three minutes of post-ictal signal. The iEEG signals were recorded intracranially with a sampling rate of either 512 Hz or 1024 Hz. All signals were down-sampled to 512 Hz for training all the models, both CA and baselines. The signals were median-referenced and band-pass filtered between 0.5 and 120 Hz using a fourth-order Butterworth filter, both in a forward and backward pass. All the recordings were inspected by an expert neurologist for identification of seizure onsets and offsets, and to remove channels corrupted by artifacts.

### Conventional Long-Term Datasets

The conventional long-term iEEG dataset contains 18 subjects, 2321 hours of recording, and 244 ictal events. Each subject is recorded continuously from implantation to explantation, regardless of the number of ictal events. The recording, post-processing, and seizure analysis setup are identical to the conventional short-term dataset.

Both datasets are publicly available and have been fully anonymized.

### Preprocessing and Task Definition

In one or more embodiments, since the datasets are already filtered, any further processing is not performed to remove artifacts and corrupted channels. As is customary in timeseries analysis, the signal is divided into samples using a certain window length and stride, which is variable depending on the model and the dataset being used. In particular, both window length and stride are subject to hyperparameter optimization as described elsewhere herein. Finally, the seizure classification task is defined as a binary classification task between ictal and non-ictal samples. A sample is considered ictal if it contains any amount of ictal activity.

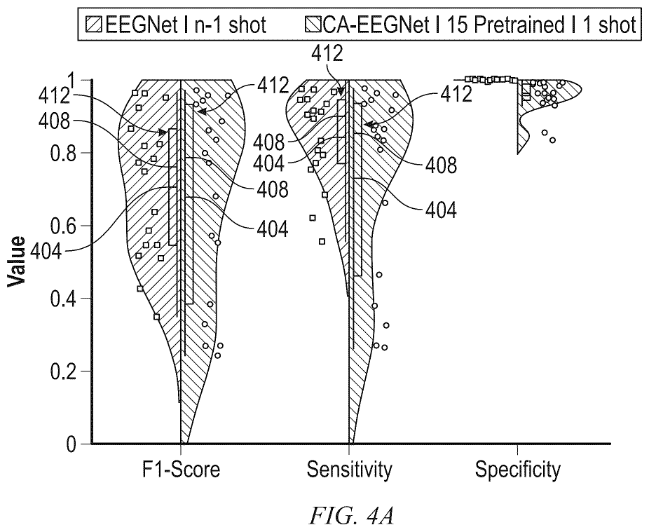

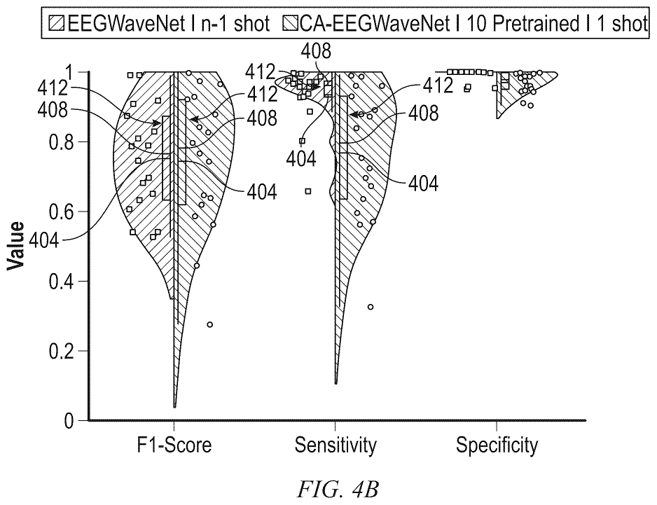

FIGS. 4A and 4B illustrate the seizure classification performance of CA models, in accordance with aspects of the invention, on the conventional long-term dataset, in accordance with exemplary embodiments. FIG. 4A shows that subject-independent CA-EEG2 (pre-trained on 15 sub-jects) surpasses the second subject-dependent conventional EEG network in F1-score. FIG. 4B shows that subject-independent CA-EEG1 (pre-trained on 10 subjects) surpasses the subject-dependent first conventional EEG network in F1-score, even when fine-tuned on a single seizure.

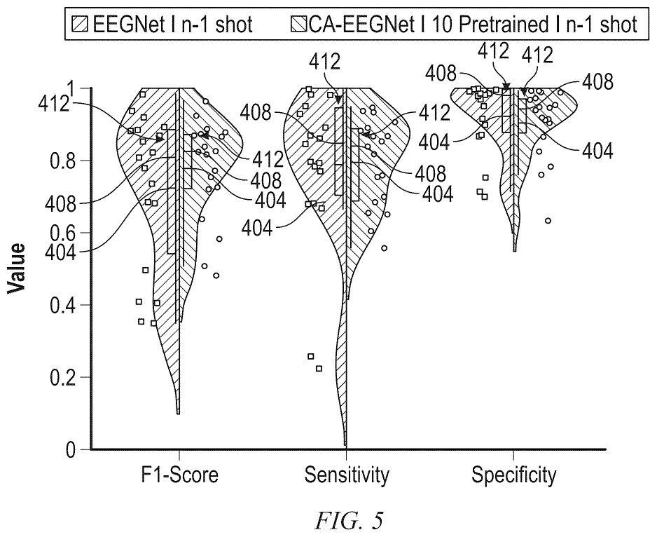

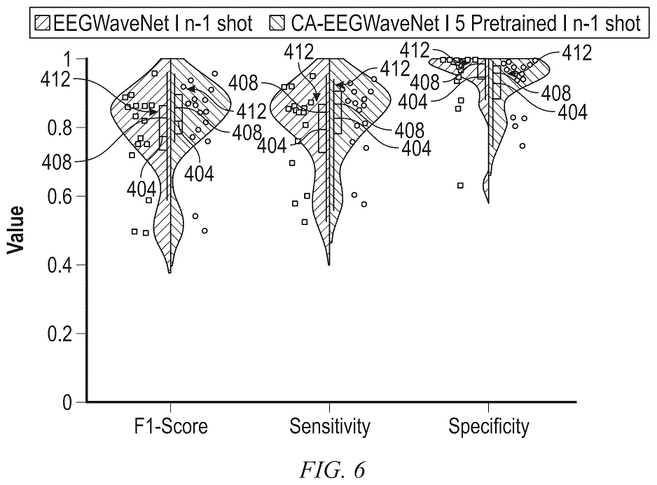

FIGS. 5-6 illustrate the seizure classification performance of the exemplary CA models on the conventional short-term dataset, in accordance with exemplary embodiments. The mean 404 (dashed line), median 408 (solid line), and quartile boxes 412 are visible. FIG. 5 shows that subject-independent CA-EEG2 (pre-trained on 10 sub-jects) surpasses the second subject-dependent conventional EEG network in F1-score. FIG. 6 shows that subject-independent CA-EEG1 (pre-trained on 5 subjects) surpasses the subject-dependent first conventional EEG network in F1-score.

### Long-Term Dataset Curation

In order to effectively train many models on the conventional long-term dataset, a subset of the non-ictal periods should be selected for each subject. A selection strategy based on the Delta power of the signal is found to be the most effective in terms of raw final performance. In particular, the power spectrum decomposition of the entire signal is computed for each subject in four second windows. The Delta power, thought to be most useful for seizure detection, is then aggregated into five bins. Finally, 20 minutes of signals are randomly taken from each bin. These 100 minutes of signal, together with all the seizures, form the available pre-training dataset.

Following this curation, the pre-training dataset is also mildly rebalanced which can improve the model's generalization. However, additional steps were purposely not taken in rebalancing, because imbalance is an intrinsic characteristic of any iEEG dataset and should be taken into account to improve clinical relevance. On the contrary, no selection using Delta power during testing was performed, to avoid biasing the model and to provide the most challenging and clinically-relevant scenario.

### Results

### Long-Term Dataset

The conventional long-term dataset is used to evaluate the exemplary CA models against a set of baselines in a clinically-relevant scenario. The recordings in this dataset are continuous and contain a large amount of variety in both ictal and interictal signals. Training follows the schema outlined elsewhere herein,” specifically regarding the curation of the dataset for the pre-training phase. In the testing phase, each seizure is presented to the model as-is together with one hour of pre-ictal and one hour of post-ictal recordings.

Given the large amount of pre-training data available, the exemplary CA architectures were fine-tuned using the LABOC scheme. This means the disclosed models receive notably less subject-specific data than the baselines, but still overall a larger amount of data from other subjects as well.

The results of CA-EEG2 pre-trained on 15 subjects against the baseline models are shown in FIG. 4A. It is worthwhile noting that CA-EEG2 achieves a median F1-score of 0.79 against 0.74 of the baseline, even when trained with only a fraction of the subject-specific data available to the baselines. This result confirms the advantage that increasing the pre-training dataset confers to the CA classifier, and highlights the generalization capabilities of the disclosed model.

The composability of the exemplary architecture and the advantages it confers by training CA-EEG1 were demonstrated. EEG-WaveNet has been shown to have increased performance on the task at hand, the same improvement is expected to transfer to CA-EEG1. CA-EEG1 was then pretrained on fewer subjects than CA-EEG2, namely 10 instead of 15, using the Long-term dataset and its performance with the first baseline conventional EEG network was compared. The results in FIG. 4B indicate that CA-EEG1 surpasses the first conventional EEG network with a median F1-score of 0.78 against 0.76.

Therefore, it has been shown that the exemplary architecture is indeed composable and that improvements in the encoder models translate to improvements of the overarching model. Moreover, the exemplary architecture also scales in the size of the dataset.

Overall, the results CA-EEG1 and CA-EEG2 achieve on the conventional long-term dataset validate the disclosed two-phase deployment. First, the base, non-personalized model can be pre-trained on a large amount of non-subject-specific data which is already available, either publicly or on an institution-by-institution basis. This initial expense, both in time and computation power required, does not need to be repeated and is offset by future use. In particular, fine-tuning CA-EEG1 and CA-EEG2 requires 5× fewer epochs than fully training a baseline model. Second, a personalized model can be quickly fine-tuned using limited amounts of subject-specific recordings, which can be more rapidly obtained in a few hours. Specifically, it was shown that a single seizure of the previously unseen subject is sufficient to achieve state-of-the-art performance, further strengthening the generalization capabilities of the exemplary CA models. As the fine-tuning time is minimal and mostly constant, the personalized model can be quickly deployed within a few minutes to hours and be put to immediate use.

### Short-Term Dataset

The conventional short-term dataset allows the evaluation of CA-EEG1 and CA-EEG2 against the baselines in a simpler scenario, where the various components are also ablated as discussed elsewhere herein. Each seizure in the Short-term dataset is accompanied by three minutes of pre-ictal and three minutes of post-ictal recording, making this a relatively balanced dataset and an ideal testing scenario. Every result is collected following the pre-training and fine-tuning schema presented elsewhere herein.

FIG. 5 shows the performance of CA-EEG2 pre-trained on ten subjects and fine-tuned with LOOC, in accordance with exemplary embodiments. CA-EEG2 surpasses its baseline conventional EEG network with a median F1-score of 0.82 against 0.80. In turn, specificity is lower, as the model is exposed to more data and this can lead to more false positives. Overall, CA-EEG2 offers better performance and faster training with ~300K less parameters.

The results of CA-EEG1 on the seizure detection task are presented in FIG. 6. Already with five subjects, pre-training CA-EEG1 outperforms its baseline both in F1-score (median of 0.85 against 0.82) and sensitivity (median of 0.87 against 0.84). It was also confirmed once more that the first conventional EEG network is a better encoder model than the second conventional EEG network, and that its performance transfers to CA-EEG1 as well.

Overall, this indicates that the exemplary architecture can be a plug-and-play replacement for different models while bringing superior performance, faster training, and better generalization capabilities.

### Component Ablation

The pertinence of each part of the exemplary pipeline are evaluated separately, both in term of training routine and model components. FIG. 3D is a table that summarizes the ablation results for each component on the short-term dataset, in accordance with exemplary embodiments. The mean (std) F1-Score, sensitivity, and specificity are reported.

### Pre-Training Phase

To evaluate the effectiveness of pre-training, CA-EEG2 was trained following the same pipeline as for the second conventional EEG network, i.e., without pre-training. As expected, pre-training is very beneficial to the model as it greatly improves the variance in the training samples and increases performance across the board.

### Fusion Component

To evaluate the impact of the fusion component 248, the evaluation pipeline was repeated on a CA-EEG2 without HRR Fusion. To achieve the combination of the channels 236, the mean of all channels 236 was taken. The performance is drastically reduced (0.45 vs 0.78), indicating that the exemplary learnable HRR Fusion is desirable to fuse the channels.

### Memory Component

To evaluate the impact of the memory component 252, the evaluation pipeline was repeated on a CA-EEG2 without TCN Memory. The fusion component 248 is fed to an MLP for final classification. The performance is drastically reduced (0.51 vs 0.78), indicating that the memory component 252 plays a pertinent role in the performance of the CA architecture.

### Subject Scaling

The exemplary CA architecture has the capability of ingesting any number of subjects for pre-training, while the baseline models need to be re-trained from scratch for every subject. Therefore, CA-EEG1 and CA-EEG2 can be pre-trained with arbitrarily large datasets to increase their generalization capabilities. At the same time, the exemplary CA models are limited in size, which might lead to sub-optimal fitting.

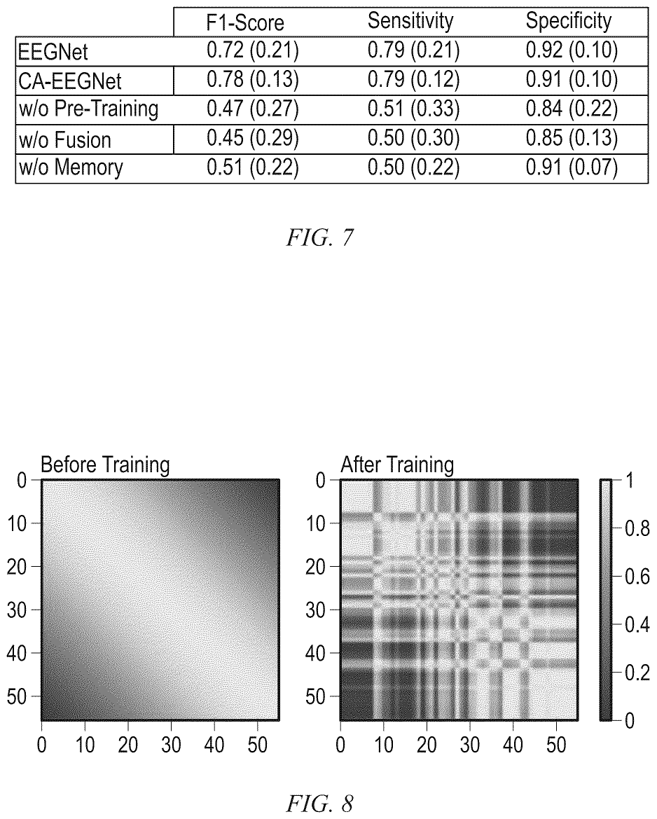

This behavior is evaluated by pre-training CA-EEG2 on the Long-term dataset with 5, 10, and 15 subjects. FIG. 7 illustrates the performance results for various versions of CA-EEG2 vs. the second conventional EEG network, in accordance with exemplary embodiments. The results in FIG. 7 confirm the considered hypothesis. Performance increases by increasing the number of subjects from 5 to 15, yielding the state-of-the-art results shown previously in FIG. 4A. However, the scaling performance is not linear, indicating more powerful encoder models may be helpful to fully take advantage of the increased dataset size. (Note that the numbers in parentheses are the standard deviations across patients.)

### Fusion Component

The fusion component 248 is pertinent to the channel-adaptive behavior of the exemplary CA architecture in one or more embodiments; therefore, how it learns the spatial structure of the signal is separately evaluated. At the beginning of training, the HRR combination vector is uniformly initialized for each subject, to represent the simplest possible inductive bias on the spatial structure. This initialization implies that nearby channels 236 are more strongly related. As training progresses, the fusion component 248 is expected to autonomously form a more nuanced understanding of the relationship between the channels 236.

This relationship was encoded as the cosine similarity between each channel's key vectors and how it evolves during training was observed. FIG. 8 clearly shows that CA-EEG2 does in fact learn information about the spatial structure of the signal after training, e.g., by forming blocks of similar channels that presumably belong to the same electrode. At the same time, the connection between some distant channels 236 is also made stronger, which is compatible with a biologically plausible explanation of different areas of the brain being more strongly connected, regardless of the distance.

It is noted that DL models commonly used for classification of EEG and iEEG, and many other multi-variate time-series, cannot successfully generalize across different subjects' clinical setups. Exemplary embodiments of the channel-adaptive classifier, which are able to process iEEG signals from any subject regardless of their clinical setup, use a learnable holographic channel fusion scheme. Exemplary embodiments show that the iEEG recordings from arbitrary subjects can be ingested by CA-EEG2 in a channel-independent encoder component 240, enabling the required generalization capabilities. By then fusing the resulting features using vector symbolic architectures, exemplary embodiments, such as CA-EEG1 and CA-EEG2, fully recover the spatial aspect of the signal. It should be understood that references herein to ‘CA-EEG2’ are to be understood as references to exemplary embodiments, and that other embodiments could differ. This capability notably enhances the exemplary CA model's clinical relevance, as the number and location of the electrodes often changes between subject and might be variable within the same subject as well, due to noise and potential deterioration of any given channel in time. Thus, exemplary embodiments are channel-adaptive.

Evidence that the exemplary model can be deployed as a one-to-one replacement of current state-of-the-art deep learning models for iEEG processing is also provided. Compared to other solutions capable of channel-adaptive classification (e.g., Transformers), the exemplary models are three to four orders of magnitude smaller (~1.2 billion (B) parameters vs ~1M), making them more practical for clinical usage. Moreover, in contrast to existing architectures, exemplary embodiments make use of a long-term memory component to store up to minutes of signal, performing the classification task on a clinically-plausible context window compatible with expert performance.

CA-EEG2 is also particularly amenable to further research and implementations, as its composable architecture is not limited to the models showcased here, and can in principle be applied to any encoder. This will allow future models to also become channel-adaptive. It is also shown that the resulting CA models are more performant and faster to train and deploy, and depending on the design of the encoder architecture, they are also smaller in size and more lightweight.

CA-EEG2 is envisioned to be of immediate practical utility in the clinic, as it can be integrated into existing pipelines to make them subject-agnostic and channel-adaptive. Currently, leveraging the vast amounts of EEG and iEEG data available to clinical institutions is challenging, as existing architectures cannot benefit from an extensive pre-training but need to be trained in a subject-specific manner. In contrast, CA-EEG2 can take advantage of any heterogeneous dataset and improve performance across the boards while bringing clinically-useful characteristics. Finally, the subject-specific training supports the development of newer and more advanced encoder models, which could be specifically designed to handle larger quantities of data and thus further aid clinical evaluation.

### Example Application

An exemplary system was used to classify seizures in the conventional long-term dataset, which contains 2321 hours of recordings, 18 patients and 244 seizures. The number of channels 236 ranges from 24 to 128. The exemplary system was pretrained on 15 patients and was then finetuned on a single seizure of the testing patient. The second baseline conventional EEG network was trained on all but one seizure of the testing patient and tested on the remaining one. A single seizure is too little to properly train the second conventional EEG network. Therefore, the system only has access to a fraction of the patient-specific data available to the baseline. Still, the system has a higher median F1-score than the baseline.

It is noted that the second baseline conventional EEG network has 2M parameters, while the tested system had 1.7M parameters. The baseline needs to be retrained from scratch for every patient, while the exemplary system needs only a fraction of the data (proportional to the number of events), and 10% of the training epochs. Overall, there is a gain of approximately two orders of magnitude in training time for each patient.

### Example Generalization to New Controller

It was demonstrated that the exemplary system can generalize to different controller networks. The first conventional EEG network was used as the controller and as the baseline. The exemplary system was pretrained on ten patients and was then finetuned on a single seizure of the testing patient. The baseline (first conventional EEG network) was trained on all but one seizure of the testing patient and tested on the remaining one. The exemplary system inherited the performance increase of the improved controller. It also improves on the performance further by increasing the median F1-score. (The first baseline conventional EEG network has 300K parameters, while the exemplary system has 1M parameters.)

### Experimental Results

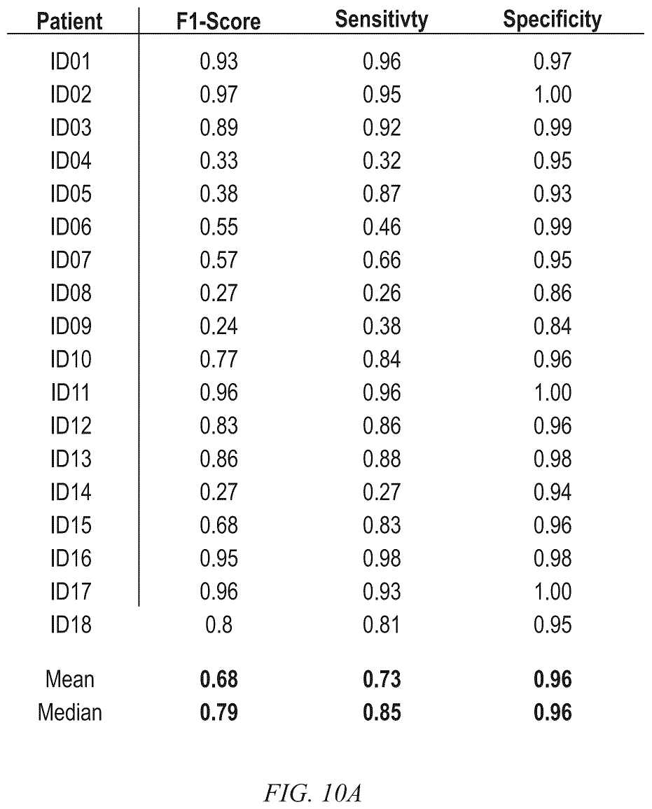

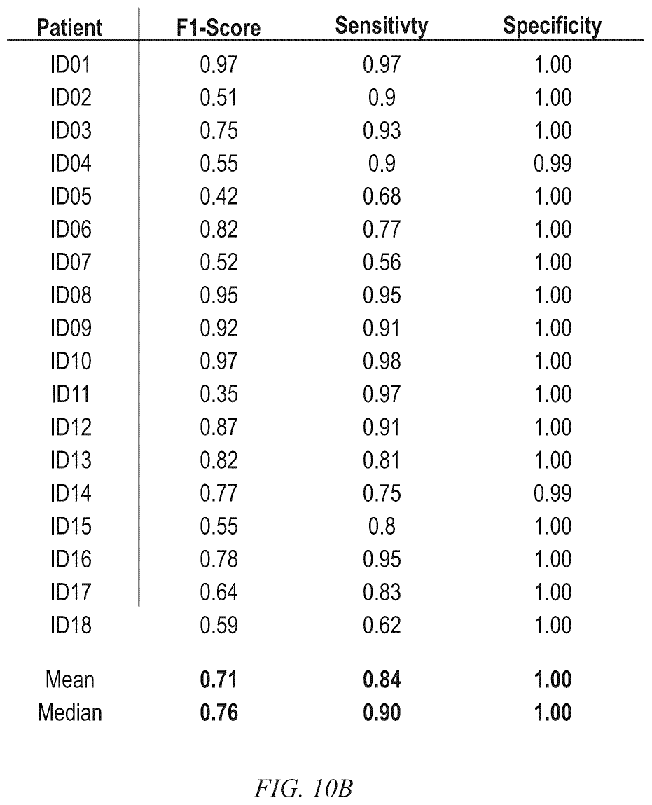

FIG. 10A shows the results on the conventional long-term dataset, in accordance with exemplary embodiments. The model was pretrained on 15 patients and finetuned on a single seizure of the testing patient.

FIG. 10B shows the results on the conventional long-term dataset, in accordance with exemplary embodiments. The model was trained on all seizures but one of the testing patient. The exemplary system performs on par despite having access to only one single seizure of the tested patient.

Refer now to FIG. 11.

Various aspects of the present disclosure are described by narrative text, flowcharts, block diagrams of computer systems and/or block diagrams of the machine logic included in computer program product (CPP) embodiments. With respect to any flowcharts, depending upon the technology involved, the operations can be performed in a different order than what is shown in a given flowchart. For example, again depending upon the technology involved, two operations shown in successive flowchart blocks may be performed in reverse order, as a single integrated step, concurrently, or in a manner at least partially overlapping in time.

A computer program product embodiment (“CPP embodiment” or “CPP”) is a term used in the present disclosure to describe any set of one, or more, storage media (also called “mediums”) collectively included in a set of one, or more, storage devices that collectively include machine readable code corresponding to instructions and/or data for performing computer operations specified in a given CPP claim. A “storage device” is any tangible device that can retain and store instructions for use by a computer processor. Without limitation, the computer readable storage medium may be an electronic storage medium, a magnetic storage medium, an optical storage medium, an electromagnetic storage medium, a semiconductor storage medium, a mechanical storage medium, or any suitable combination of the foregoing. Some known types of storage devices that include these mediums include: diskette, hard disk, random access memory (RAM), read-only memory (ROM), erasable programmable read-only memory (EPROM or Flash memory), static random access memory (SRAM), compact disc read-only memory (CD-ROM), digital versatile disk (DVD), memory stick, floppy disk, mechanically encoded device (such as punch cards or pits/lands formed in a major surface of a disc) or any suitable combination of the foregoing. A computer readable storage medium, as that term is used in the present disclosure, is not to be construed as storage in the form of transitory signals per se, such as radio waves or other freely propagating electromagnetic waves, electromagnetic waves propagating through a waveguide, light pulses passing through a fiber optic cable, electrical signals communicated through a wire, and/or other transmission media. As will be understood by those of skill in the art, data is typically moved at some occasional points in time during normal operations of a storage device, such as during access, de-fragmentation or garbage collection, but this does not render the storage device as transitory because the data is not transitory while it is stored.

Computing environment 100 contains an example of an environment for the execution of at least some of the computer code involved in performing the inventive methods, such as VSA-based system 200. In addition to block 200, computing environment 100 includes, for example, computer 101, wide area network (WAN) 102, end user device (EUD) 103, remote server 104, public cloud 105, and private cloud 106. In this embodiment, computer 101 includes processor set 110 (including processing circuitry 120 and cache 121), communication fabric 111, volatile memory 112, persistent storage 113 (including operating system 122 and block 200, as identified above), peripheral device set 114 (including user interface (UI) device set 123, storage 124, and Internet of Things (IoT) sensor set 125), and network module 115. Remote server 104 includes remote database 130. Public cloud 105 includes gateway 140, cloud orchestration module 141, host physical machine set 142, virtual machine set 143, and container set 144.

COMPUTER 101 may take the form of a desktop computer, laptop computer, tablet computer, smart phone, smart watch or other wearable computer, mainframe computer, quantum computer or any other form of computer or mobile device now known or to be developed in the future that is capable of running a program, accessing a network or querying a database, such as remote database 130. As is well understood in the art of computer technology, and depending upon the technology, performance of a computer-implemented method may be distributed among multiple computers and/or between multiple locations. On the other hand, in this presentation of computing environment 100, detailed discussion is focused on a single computer, specifically computer 101, to keep the presentation as simple as possible. Computer 101 may be located in a cloud, even though it is not shown in a cloud in FIG. 11. On the other hand, computer 101 is not required to be in a cloud except to any extent as may be affirmatively indicated.

PROCESSOR SET 110 includes one, or more, computer processors of any type now known or to be developed in the future. Processing circuitry 120 may be distributed over multiple packages, for example, multiple, coordinated integrated circuit chips. Processing circuitry 120 may implement multiple processor threads and/or multiple processor cores. Cache 121 is memory that is located in the processor chip package(s) and is typically used for data or code that should be available for rapid access by the threads or cores running on processor set 110. Cache memories are typically organized into multiple levels depending upon relative proximity to the processing circuitry. Alternatively, some, or all, of the cache for the processor set may be located “off chip.” In some computing environments, processor set 110 may be designed for working with qubits and performing quantum computing.

Computer readable program instructions are typically loaded onto computer 101 to cause a series of operational steps to be performed by processor set 110 of computer 101 and thereby effect a computer-implemented method, such that the instructions thus executed will instantiate the methods specified in flowcharts and/or narrative descriptions of computer-implemented methods included in this document (collectively referred to as “the inventive methods”). These computer readable program instructions are stored in various types of computer readable storage media, such as cache 121 and the other storage media discussed below. The program instructions, and associated data, are accessed by processor set 110 to control and direct performance of the inventive methods. In computing environment 100, at least some of the instructions for performing the inventive methods may be stored in block 200 in persistent storage 113.

COMMUNICATION FABRIC 111 is the signal conduction path that allows the various components of computer 101 to communicate with each other. Typically, this fabric is made of switches and electrically conductive paths, such as the switches and electrically conductive paths that make up busses, bridges, physical input/output ports and the like. Other types of signal communication paths may be used, such as fiber optic communication paths and/or wireless communication paths.

VOLATILE MEMORY 112 is any type of volatile memory now known or to be developed in the future. Examples include dynamic type random access memory (RAM) or static type RAM. Typically, volatile memory 112 is characterized by random access, but this is not required unless affirmatively indicated. In computer 101, the volatile memory 112 is located in a single package and is internal to computer 101, but, alternatively or additionally, the volatile memory may be distributed over multiple packages and/or located externally with respect to computer 101.

PERSISTENT STORAGE 113 is any form of non-volatile storage for computers that is now known or to be developed in the future. The non-volatility of this storage means that the stored data is maintained regardless of whether power is being supplied to computer 101 and/or directly to persistent storage 113. Persistent storage 113 may be a read only memory (ROM), but typically at least a portion of the persistent storage allows writing of data, deletion of data and re-writing of data. Some familiar forms of persistent storage include magnetic disks and solid state storage devices. Operating system 122 may take several forms, such as various known proprietary operating systems or open source Portable Operating System Interface-type operating systems that employ a kernel. The code included in block 200 typically includes at least some of the computer code involved in performing the inventive methods.

PERIPHERAL DEVICE SET 114 includes the set of peripheral devices of computer 101. Data communication connections between the peripheral devices and the other components of computer 101 may be implemented in various ways, such as Bluetooth connections, Near-Field Communication (NFC) connections, connections made by cables (such as universal serial bus (USB) type cables), insertion-type connections (for example, secure digital (SD) card), connections made through local area communication networks and even connections made through wide area networks such as the internet. In various embodiments, UI device set 123 may include components such as a display screen, speaker, microphone, wearable devices (such as goggles and smart watches), keyboard, mouse, printer, touchpad, game controllers, and haptic devices. Storage 124 is external storage, such as an external hard drive, or insertable storage, such as an SD card. Storage 124 may be persistent and/or volatile. In some embodiments, storage 124 may take the form of a quantum computing storage device for storing data in the form of qubits. In embodiments where computer 101 is required to have a large amount of storage (for example, where computer 101 locally stores and manages a large database) then this storage may be provided by peripheral storage devices designed for storing very large amounts of data, such as a storage area network (SAN) that is shared by multiple, geographically distributed computers. IoT sensor set 125 is made up of sensors that can be used in Internet of Things applications. For example, one sensor may be a thermometer and another sensor may be a motion detector.

NETWORK MODULE 115 is the collection of computer software, hardware, and firmware that allows computer 101 to communicate with other computers through WAN 102. Network module 115 may include hardware, such as modems or Wi-Fi signal transceivers, software for packetizing and/or de-packetizing data for communication network transmission, and/or web browser software for communicating data over the internet. In some embodiments, network control functions and network forwarding functions of network module 115 are performed on the same physical hardware device. In other embodiments (for example, embodiments that utilize software-defined networking (SDN)), the control functions and the forwarding functions of network module 115 are performed on physically separate devices, such that the control functions manage several different network hardware devices. Computer readable program instructions for performing the inventive methods can typically be downloaded to computer 101 from an external computer or external storage device through a network adapter card or network interface included in network module 115.

WAN 102 is any wide area network (for example, the internet) capable of communicating computer data over non-local distances by any technology for communicating computer data, now known or to be developed in the future. In some embodiments, the WAN 102 may be replaced and/or supplemented by local area networks (LANs) designed to communicate data between devices located in a local area, such as a Wi-Fi network. The WAN and/or LANs typically include computer hardware such as copper transmission cables, optical transmission fibers, wireless transmission, routers, firewalls, switches, gateway computers and edge servers.

END USER DEVICE (EUD) 103 is any computer system that is used and controlled by an end user (for example, a customer of an enterprise that operates computer 101), and may take any of the forms discussed above in connection with computer 101. EUD 103 typically receives helpful and useful data from the operations of computer 101. For example, in a hypothetical case where computer 101 is designed to provide a recommendation to an end user, this recommendation would typically be communicated from network module 115 of computer 101 through WAN 102 to EUD 103. In this way, EUD 103 can display, or otherwise present, the recommendation to an end user. In some embodiments, EUD 103 may be a client device, such as thin client, heavy client, mainframe computer, desktop computer and so on.

REMOTE SERVER 104 is any computer system that serves at least some data and/or functionality to computer 101. Remote server 104 may be controlled and used by the same entity that operates computer 101. Remote server 104 represents the machine(s) that collect and store helpful and useful data for use by other computers, such as computer 101. For example, in a hypothetical case where computer 101 is designed and programmed to provide a recommendation based on historical data, then this historical data may be provided to computer 101 from remote database 130 of remote server 104.

PUBLIC CLOUD 105 is any computer system available for use by multiple entities that provides on-demand availability of computer system resources and/or other computer capabilities, especially data storage (cloud storage) and computing power, without direct active management by the user. Cloud computing typically leverages sharing of resources to achieve coherence and economies of scale. The direct and active management of the computing resources of public cloud 105 is performed by the computer hardware and/or software of cloud orchestration module 141. The computing resources provided by public cloud 105 are typically implemented by virtual computing environments that run on various computers making up the computers of host physical machine set 142, which is the universe of physical computers in and/or available to public cloud 105. The virtual computing environments (VCEs) typically take the form of virtual machines from virtual machine set 143 and/or containers from container set 144. It is understood that these VCEs may be stored as images and may be transferred among and between the various physical machine hosts, either as images or after instantiation of the VCE. Cloud orchestration module 141 manages the transfer and storage of images, deploys new instantiations of VCEs and manages active instantiations of VCE deployments. Gateway 140 is the collection of computer software, hardware, and firmware that allows public cloud 105 to communicate through WAN 102.

Some further explanation of virtualized computing environments (VCEs) will now be provided. VCEs can be stored as “images.” A new active instance of the VCE can be instantiated from the image. Two familiar types of VCEs are virtual machines and containers. A container is a VCE that uses operating-system-level virtualization. This refers to an operating system feature in which the kernel allows the existence of multiple isolated user-space instances, called containers. These isolated user-space instances typically behave as real computers from the point of view of programs running in them. A computer program running on an ordinary operating system can utilize all resources of that computer, such as connected devices, files and folders, network shares, CPU power, and quantifiable hardware capabilities. However, programs running inside a container can only use the contents of the container and devices assigned to the container, a feature which is known as containerization.

PRIVATE CLOUD 106 is similar to public cloud 105, except that the computing resources are only available for use by a single enterprise. While private cloud 106 is depicted as being in communication with WAN 102, in other embodiments a private cloud may be disconnected from the internet entirely and only accessible through a local/private network. A hybrid cloud is a composition of multiple clouds of different types (for example, private, community or public cloud types), often respectively implemented by different vendors. Each of the multiple clouds remains a separate and discrete entity, but the larger hybrid cloud architecture is bound together by standardized or proprietary technology that enables orchestration, management, and/or data/application portability between the multiple constituent clouds. In this embodiment, public cloud 105 and private cloud 106 are both part of a larger hybrid cloud.

The descriptions of the various embodiments of the present invention have been presented for purposes of illustration, but are not intended to be exhaustive or limited to the embodiments disclosed. Many modifications and variations will be apparent to those of ordinary skill in the art without departing from the scope and spirit of the described embodiments. The terminology used herein was chosen to best explain the principles of the embodiments, the practical application or technical improvement over technologies found in the marketplace, or to enable others of ordinary skill in the art to understand the embodiments disclosed herein.

What is claimed is:

1. A computer-implemented method comprising:
encoding a plurality of channels to provide short-term feature extraction from multi-channel timeseries data for each of a plurality of short-term windows to generate channel-by-channel feature vectors and provide a structured latent space representation of the plurality of channels;
fusing the channel-by-channel feature vectors to incorporate an original spatial structure of the plurality of channels into a single short-term representation for each of the plurality of short-term windows;
extracting long-term temporal features from the single short-term representations;
performing long-term context processing on the long-term temporal features;
classifying the multi-channel timeseries data based on results of the long-term processing; and
outputting a result of the classification.

2. The method of claim 1, wherein the extracting of the long-term temporal features comprises extracting the long-term features by extending an effective receptive field over the plurality of short-term windows.

3. The method of claim 1, further comprising splitting each of the plurality of channels into the plurality of short-term windows in a time and space dimensions prior to the encoding of the plurality of channels.

4. The method of claim 1, wherein the encoding of each channel is performed in parallel and independently.

5. The method of claim 1, wherein the fusing is enabled by holographic reduced representations (HRR) where only a single parameter per channel is used to encode a corresponding channel setup.

6. The method of claim 1, wherein the fusing combines channel-wise features while preserving spatial information between the plurality of channels.

7. The method of claim 1, further comprising aggregating the plurality of short-term representations into a single long-term representation of a long-term context window, and wherein the extracting of the long-term temporal features is based on the single long-term representation.

8. The method of claim 1, wherein the fusing of the channel-by-channel feature vectors further comprises preserving a relationship between the channels as an angular distance between components of the single representation.

9. The method of claim 1, further comprising:
detecting a medical condition of a patient based on the classifying of the multi-channel timeseries data, wherein the multi-channel timeseries data is biometric data; and
implementing a medical therapy based on the detected medical condition.

10. The method of claim 9, wherein the implementing of the medical therapy further comprises performing at least one of automatically delivering a drug therapy based on the detected medical condition and automatically administering responsive neurostimulation based on the detected medical condition.

11. The method of claim 1, wherein the long-term context processing is performed using a temporal convolutional network.

12. A computer program product, comprising:
one or more tangible computer-readable storage media and program instructions stored on at least one of the one or more tangible computer-readable storage media, the program instructions executable by a processor, the program instructions comprising:
encoding a plurality of channels to provide short-term feature extraction from multi-channel timeseries data for each of a plurality of short-term windows to generate channel-by-channel feature vectors and provide a structured latent space representation of the plurality of channels;
fusing the channel-by-channel feature vectors to incorporate an original spatial structure of the plurality of channels into a single short-term representation for each of the plurality of short-term windows;
extracting long-term temporal features from the single short-term representations;
performing long-term context processing on the long-term temporal features;
classifying the multi-channel timeseries data based on results of the long-term processing; and
outputting a result of the classification.

13. A system comprising:
a memory; and
at least one processor, coupled to said memory, and operative to perform operations comprising:
encoding a plurality of channels to provide short-term feature extraction from multi-channel timeseries data for each of a plurality of short-term windows to generate channel-by-channel feature vectors and provide a structured latent space representation of the plurality of channels;
fusing the channel-by-channel feature vectors to incorporate an original spatial structure of the plurality of channels into a single short-term representation for each of the plurality of short-term windows;
extracting long-term temporal features from the single short-term representations;
performing long-term context processing on the long-term temporal features;
classifying the multi-channel timeseries data based on results of the long-term processing; and
outputting a result of the classification.

14. The system of claim 13, wherein the extracting of the long-term temporal features comprises extracting the long-term features by extending an effective receptive field over the plurality of short-term windows.

15. The system of claim 13, wherein the operations performed by the at least one processor further comprise splitting each of the plurality of channels into the plurality of short-term windows in a time and space dimensions prior to the encoding of the plurality of channels.

16. The system of claim 13, wherein the fusing is enabled by holographic reduced representations (HRR) where only a single parameter per channel is used to encode a corresponding channel setup.

17. The system of claim 13, wherein the fusing combines channel-wise features while preserving spatial information between the plurality of channels.

18. The system of claim 13, wherein the operations performed by the at least one processor further comprise aggregating the plurality of short-term representations into a single long-term representation of a long-term context window, and wherein the extracting of the long-term temporal features is based on the single long-term representation.

19. The system of claim 13, wherein the fusing of the channel-by-channel feature vectors further comprises preserving a relationship between the channels as an angular distance between components of the single representation.

20. The system of claim 13, wherein the operations performed by the at least one processor further comprise:
detecting a medical condition of a patient based on the classifying of the multi-channel timeseries data, wherein the multi-channel timeseries data is biometric data; and
implementing a medical therapy based on the detected medical condition.
